(19) United States
(12) Patent Application Publication
(10) Pub. No.: US 2023/0263994 A1
(43) Pub. Date: Aug. 24, 2023

(54) CATHETER FOR FORMING AN INTERVASCULAR BRIDGE TO RELIEVE VESSEL COMPRESSION

(71) Applicant: C.R. BARD, INC., Franklin Lakes, NJ (US)

(72) Inventors: Matt Casiraro, Tempe, AZ (US); Casey Rockwood, Tempe, AZ (US)

(21) Appl. No.: 18/020,385

(22) PCT Filed: Aug. 13, 2020
(86) PCT No.: PCT/US2020/046073

§ 371 Date: Feb. 8, 2023

(51) Int. Cl. A61M 25/00; A61M 25/09; A61M 25/10

(52) U.S. Cl. CPC A61M 25/0084; A61M 25/09; A61M 25/1018; A61M 2205/0266

(57) ABSTRACT

A catheter (10) is provided for relieving compression among blood vessels by introducing a filler into an intravascular space to form a bridge connecting the vessels in order to relieve the compression. The catheter is provided with a shaft (14), expandable elements (16,18) supported by the shaft for reliving compression in the vessel temporarily, and a retractable needle (30) located between the expandable elements. When deployed, such as by using an actuator (40), the needle may deliver the filler to the intervascular space to form a bridge between the vessels for relieving the compression once the catheter is removed.

### INCORPORATION BY REFERENCE

All publications and patent applications mentioned in this specification are herein incorporated by reference to the same extent as if each individual publication or patent application was specifically and individually indicated to be incorporated by reference.

### BACKGROUND

A condition known as May-Thurner Syndrome is caused when the left iliac vein LIV is compressed by the right iliac artery RIA (see cross-sectional view provided in FIG. 1, illustrating location of these vessels relative to the right iliac vein RIV, left iliac artery LIA, and an adjacent (typically fifth lumbar) vertebra V). This condition makes it more likely for deep vein thrombosis to occur, which can have deleterious consequences.

Current treatments for treating vessel compression involve surgical repair, which is highly invasive and undesirable, or by attempting to restore patency to the left iliac vein using an endovascular implant (e.g., a stent). While the endovascular procedure is preferred over surgery in many cases due to the lack of invasiveness, it requires leaving the implant in the vein to relieve the compression. This can sometimes create issues, such as creating thromboembolisms, discomfort, or even collapse.

Accordingly, a need is identified for an improved manner of relieving compression among vessels, such as for treating May-Thurner Syndrome. Specifically, there is a need for an improved manner of relieving compression among vessels which avoids leaving an endovascular implant in the body.

### SUMMARY

In one aspect, this disclosure relates to a catheter including a shaft. Expandable elements are supported by the shaft, and when expanded may serve to relieve compression by expanding the associated vessel. A needle located between the expandable elements may be deployed and used to deliver a filler to an intervascular space for forming a bridge, which may thus form a connection between the vessels and help to more permanently relieve the compression once the expandable elements are deflated and removed from the vessel.

In one embodiment, the shaft includes a lumen, and the needle is hollow and in communication with the lumen. The needle may be retractable within the lumen, and may be deployed by way of an actuator. The shaft may further include an inflation lumen, and the expandable elements comprise inflatable balloons in fluid communication with the inflation lumen.

In use, the balloons are inflated to open up a compressed or pinched vessel (such as a vein compressed due to May-Thurner Syndrome). A filler may be delivered via the deployed needle while balloons are inflated and then hardens (e.g., within three minutes). Once the filler is cured or hardened, the balloons can be deflated and the resultant vessel is opened or uncompressed as it has been sealed externally along its arc length to an adjacent vessel. The shaft may further comprise a guidewire lumen in addition to the lumen for filler delivery, which may be used for placing the catheter in the vessel via a guidewire.

A plurality of needles may be located between the expandable elements, such as for injecting material on different sides of a targeted vessel. The needle(s) may be curved when deployed, and may comprise a shape memory material that, as a result of temperature conditions, may assume a particular orientation when the shape memory is activated. The filler, such as an adhesive, may be supplied to the catheter from a remote source for delivery to a location in an intervascular space via the needle.

According to a further aspect of the disclosure, a system for relieving compression among blood vessels with an adjacent intervascular space is provided. The system comprises a catheter including a needle adapted for extending into the intervascular space when deployed. The system further includes a filler for delivery via the needle into the intravascular space.

In one version, the catheter comprises spaced apart expandable elements, and the needle is located between the expandable elements when deployed. The expandable elements may comprise inflatable balloons. The needle may be curved when deployed, and may comprise a shape memory material. The needle may be deploying using an actuator and a plurality of needles may be provided for delivering filler to different locations in the intervascular space.

This disclosure also pertains to a method of relieving compression among blood vessels. The method comprises injecting a filler into an intervascular space between the blood vessels. The injecting step may comprise injecting the filler to the intervascular space using a needle connected to a catheter. Prior to the injecting step, the method may involve inserting the catheter into one of the blood vessels, expanding expandable elements on the catheter, and inserting the needle through a vessel wall and into the intervascular space. The injecting step may comprise deploying the needle from a retracted position within the catheter to a space between the expandable elements.

### BRIEF DESCRIPTION OF THE DRAWINGS

The above and further advantages of the disclosure may be better understood by referring to the following description in conjunction with the accompanying drawings in which:

FIG. 1 is a cross-sectional view of an environment of use of the proposed invention in the human vasculature.

FIG. 2 is a side schematic view of a catheter forming one aspect of the disclosure.

FIGS. 2A and 2B are cross-sectional views of the catheter of FIG. 2 taken along lines 2A-2A and 2B-2B of FIG. 2, respectively.

FIG. 3 is a side schematic view of a catheter forming one aspect of the disclosure.

FIG. 3A is a cross-sectional view of the catheter of FIG. 3, taken along line 3A-3A of FIG. 3.

FIGS. 4 and 5 illustrate various actuators for deploying the needle(s) to enter the intervascular space.

FIGS. 6,7, 8, 9, 10, and 11 are progressive views of the steps used to treat vessel compression by creating a bridge using a catheter according to one aspect of the disclosure.

FIG. 12 is a cross-sectional view of the vascular environment showing the location of the bridge for relieving intervascular compression.

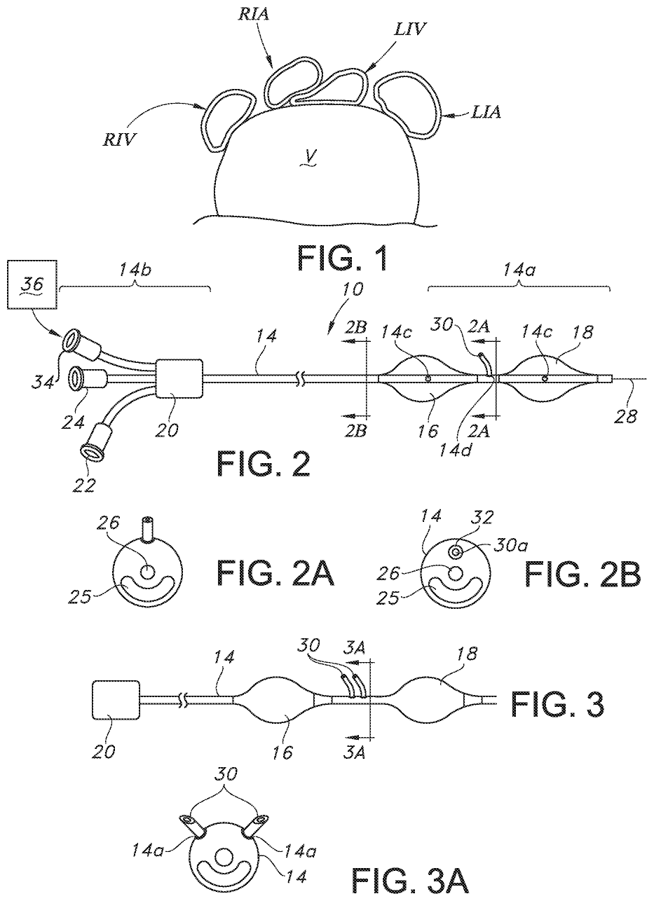

The dimensions of some of the elements may be exaggerated relative to other elements for clarity or several physical components may be included in one functional block or element. Further, sometimes reference numerals may be repeated among the drawings to indicate corresponding or analogous elements. Moreover, some of the items depicted in the drawings may be combined into a single function.

### DETAILED DESCRIPTION

The present disclosure provides a catheter and related methods for forming a bridge in an intervascular space, which may be used to relieve compression among vessels, and aims to remove the need to implant an endovascular device or for surgical intervention. The catheter would be capable of expanding a compressed vessel into which it is inserted, such as by using expandable elements, and then injecting a filler into the intervascular space using one or more needles deployed between the expandable elements. The bridge serves to maintain the compression relief, even after the catheter is withdrawn.

In the following detailed description, numerous specific details are set forth to provide a thorough understanding of the present invention. The disclosed embodiments may be practiced without these specific details. In other instances, well-known methods, procedures, components, or structures may not have been described in detail so as not to obscure the aspects of the present disclosure.

The present disclosure is directed to systems and methods for treatment of a vessel. The principles and operation of systems and methods of the disclosure may be better understood with reference to the drawings and accompanying descriptions.

The invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein are for the purpose of description and should not be regarded as limiting.

Certain features of the invention that are, for clarity, described in the context of separate embodiments, may also be provided in combination in a single embodiment. Conversely, various features of the invention that are, for brevity, described in the context of a single embodiment, may also be provided separately or in any suitable sub-combination.

With reference to FIG. 2, a catheter 10 is provided for intervascular use. The catheter comprises a shaft 14 with a distal end portion 14a supporting at least two expandable elements. The expandable elements may take the form of inflatable balloons 16, 18 positioned in tandem. The balloons 16, 18 may be of the type typically used for percutaneous transluminal angioplasty, but in this use are not designed to compress a plaque, but rather simply expand to a diameter similar to that of a vessel into which the balloons are located to relieve compression, and possibly to provide an anchoring and locating function, as will be understood further upon reviewing the following description.

The proximal end portion 14b of the shaft 14 may include a hub 20 which includes an inflation port 22 for delivering an inflation fluid to the balloons 16, 18 via an inflation lumen 25 in the shaft 14 (which includes ports or openings 14c communicating with the interior compartments of the balloons). The hub 20 may also include a guidewire port 24 communicating with a guidewire lumen 26 in the shaft 14. The guidewire lumen 26 may receive a guidewire 28 for guiding the catheter 10 to an endovascular location, such as the junction of the left iliac vein and the right iliac artery being compressed.

At least one needle 30 may be connected to the shaft 14 for deployment from a retracted location between the expansion elements, such as balloons 16, 18. The needle 30 is hollow, and is adapted to project transversely to an extent greater than the maximum diameter of the balloons 16, 18 when inflated in a vessel to cause a tip of the needle located between the balloons 16, 18 to perforate or puncture a wall of the vessel and enter an intervascular space (that is, a space located outside of a vessel, and adjacent to another vessel). More specifically, the needle 30 may initially be retracted into a lumen 32 of the shaft 14, and may extend from within the shaft 14 via an opening or port 14c between the balloons 16, 18, such that a distal tip thereof enters the intervascular space. Deployment of the needle 30 may be done from a remote location, such as outside of the body into which the catheter 10 is introduced. In a simple example, the needle 30 may simply include an elongated tube 30a, which passes through the lumen 32 to adjacent the hub 20. The tube 30a may then be advanced from adjacent the hub 20 to move within the shaft 14 and cause the needle 30 to deploy (which may be guided in part by gradually curving the distal end of the lumen 32 toward the opening or port 14c).

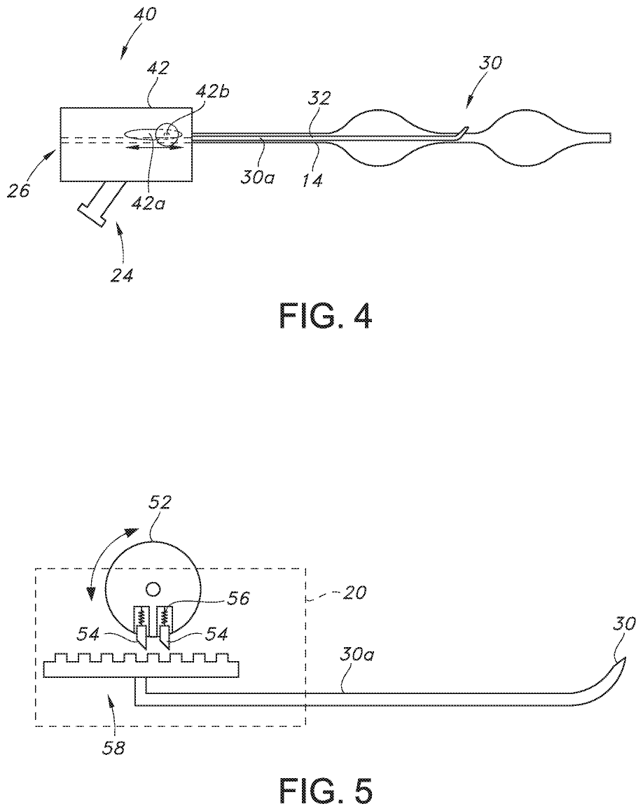

Alternatively, the tube 30a may be connected to an actuator 40 for advancing and retracting the tube 30a, and hence the needle 30, relative to the shaft 14. For example, in FIG. 4, a proximal end of the tube 30a may be connected to a slide block 42 having a slot 42a for engaging a transverse post 42b connected to the tube 30a. The block 42 may also include a guidewire lumen 26, as well as the port 24 for communicating with inflation lumen. Movement of the post 42b within the block 42 in the distal direction thus serves to deploy the needle 30 into the intervascular space, and the opposite movement in the proximal direction serves to retract the needle to within the shaft 14 for removal from the vessel. The length of slot 42a may define the maximum distance of deployment, which may correspond to the maximum diameter of the balloons 16, 18 plus the added extension needed to cause the needle tip to enter the intervascular space.

An alternative actuator 50 is shown in FIG. 5. This embodiment uses a ratchet and pawl type of arrangement, whereby a thumb wheel 52 includes pawls 54 that are biased by springs 56 into engagement with a linear actuator or rack 58. The rack 58 is in turn connected to the tube 30a, which is located within the lumen 32 of shaft 14. Rotation of the wheel 52 thus causes the rack 58 and, hence, the tube 30a, to move, such as in the distal direction to deploy the needle 30 into the intervascular space, and then to retract again. The actuator 50 may form part of the hub 20, as indicated in phantom lines, or may comprise a separate structure.

The needle 30 may optionally be fabricated of a shape memory material, such as Nitinol The shape memory may be created during fabrication, and may be activated at a particular temperature (which may be regulated, for example, by the clinician introducing a fluid at a temperature for activating the shape memory, and thus causing the needle to assume a particular preset shape or orientation). When activated by body temperature and advanced out of a straight configuration from within the shaft 14, the needle 30 will stiffen and take its pre-set shape. For example, the needle 30 may assume a pre-set curved configuration and project radially outwardly from the shaft 14, as shown.

When deployed, the needle 30 is placed in communication with a remote port 34 outside the body, such as by way of connection to the tube 30a. This remote port 34 is connected to a source 36 of a filler, such as an adhesive (such as, for example, Tridyne, cyanoacrylate, natural chitosan adhesive, or the like). The source 36 may comprise a syringe or other delivery device for delivering the filler to the lumen 32 under pressure for causing the material to flow along the tube 30a located in lumen 32 and exit a tip of the needle 30 when deployed between the balloons 16, 18, and thus enter the intervascular space at a location corresponding to the vessel-to-vessel compression.

FIGS. 3 and 3A illustrate that two or more needles 30 may be provided between the expandable elements 16, 18, for projecting in different directions when deployed (which may be done in the manner discussed above, using a single actuator or separate actuators). The provision of two or more needles 30 allows for the filler material delivery to different locations in the intervascular space, such as different sides of the compressed vessel into which the catheter 10 is inserted. The needles 30 may extend in different radial directions, as perhaps best understood in FIG. 3A, and may be retracted within the shaft 14 and, when deployed, project from openings 14a spaced in the longitudinal direction along the shaft 14 of catheter 10. Both needles 30 may communicate with associated lumens for delivering a filler from a remote source, as with the single needle embodiment.

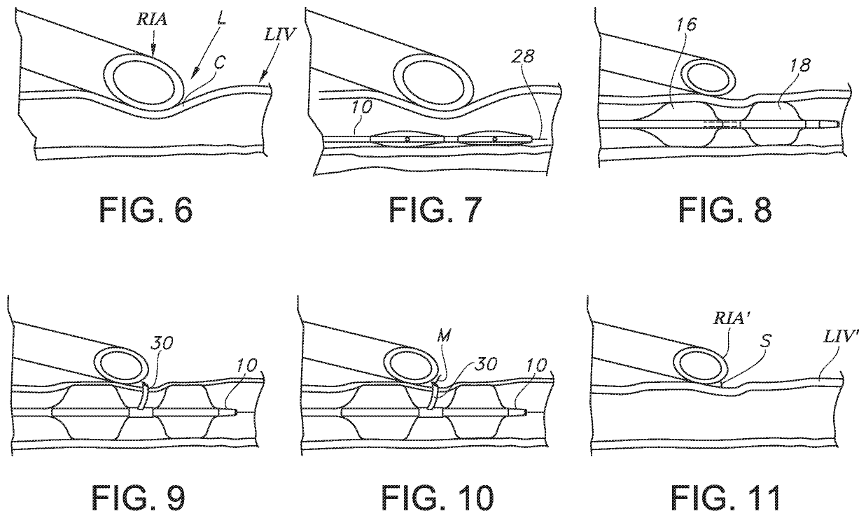

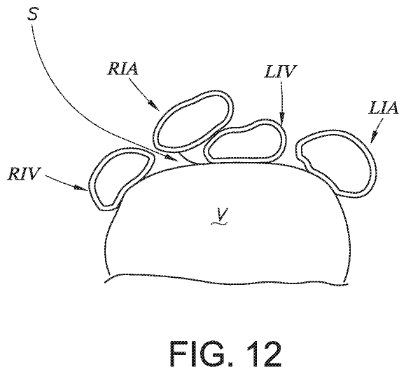

In use, the catheter 10 may be tracked via guidewire 28 to a location L of a compression C of a vessel, such as the left iliac vein LIV as shown in FIG. 4 being compressed by the right iliac artery RIA, as is characteristic of May-Thurner Syndrome. Once the catheter 10 is positioned at the location (which may be achieved with the aid of radiopaque markers on the catheter 10 and the use of fluoroscopy), as indicated in FIG. 5, the balloons 16, 18 are expanded, which serves to expand the compressed vessel and anchor the catheter in position (see FIG. 6). The position is such that, when the needle 30 is deployed, as shown in FIG. 7, and, when activated via the shape memory, the tip of it enters the intervascular space.

Filler material M may then be delivered from the source 36 via tube 30a located within lumen 32. This material enters the intervascular space via needle 30 (and, in the case of two or more needles, the material may be delivered simultaneously to two different locations, such as different sides of the compressed vessel). Once a sufficient amount of filler material M is delivered, which may be done by volume, time, and/or under fluoroscopy to observe the amount of material ejected from the needle 30, and the material cures or hardens (which may happen fairly rapidly, e.g. less than three minutes from injection) the balloons 16, 18 may be deflated, and the catheter 10 withdrawn.

The material M once dried cured forms a bridge S external to and between the vessels LIV, RIA, as perhaps best understood in FIG. 10, and may further serve to connect or bond the adjacent vessels to each other. In any case, the bridge S serves to remove the compression that previously existed (note patent left Iliac vein LIV in FIG. 9), even after the catheter 10 is removed. Consequently, a more patent venous system is achieved, was may provide relief from the effects of May-Thurner Syndrome, without surgical intervention and without the use of an endovascular implant.

Summarizing, this disclosure may be considered to relate to the following items:

1. A catheter for relieving compression among blood vessels by introducing a filler into an intravascular space, comprising:

a shaft;

expandable elements supported by the shaft; and

a needle supported by the shaft, the needle, when deployed, including a tip located between the expandable elements for introducing the filter into the intravascular space.

2. The catheter of item 1, wherein the shaft includes a lumen, and the needle is hollow.
3. The catheter of item 1, item 2 or any of items 4-9 following hereafter, further including an actuator for deploying the needle from a retracted position within the lumen to place the tip between the expandable elements and into the intervascular space for delivery of the filler.
4. The catheter of any of items 1-3, wherein the shaft includes an inflation lumen, and the expandable elements comprise inflatable balloons in fluid communication with the inflation lumen.
5. The catheter of any of items 1-4, wherein the shaft comprises a guidewire lumen.
6. The catheter of any of items 1-5, wherein a plurality of needles, when deployed, have tips located between the expandable elements.
7. The catheter of any of items 1-6, wherein the needle is/needles are curved.
8. The catheter of item 1, wherein the needle comprises a shape memory material.
9. The catheter of item 1, further including a filler for delivery to a location in an intervascular space via the needle.
10. A system for relieving compression among blood vessels with an adjacent intervascular space, comprising:

a catheter including a retractable needle adapted for extending into the intervascular space when deployed; and

a filler for delivery via the needle into the intravascular space.

11. The system of item 10, wherein the catheter comprises spaced apart expandable elements, the needle located between the expandable elements when deployed.
12. The system of item 10 or item 11, wherein the expandable elements comprise inflatable balloons.
13. The system of any of items 11-12, wherein the needle is curved when deployed.
14. The system of any of items 11-13, wherein the needle comprises a shape memory material.
15. The system of any of items 11-14, further including an actuator for moving the needle from a retracted position within the catheter to a deployed position for delivering the filler to the intervascular space.
16. The system of any of items 11-15, wherein the catheter includes a plurality of needles and, optionally, an actuator for deploying each needle.
17. A method of relieving compression among blood vessels, comprising:

injecting a filler into an intervascular space between the blood vessels.

18. The method of claim 17, wherein the injecting step comprises injecting the filler to the intervascular space using a needle connected to a catheter.
19. The method of item 17 or item 18, wherein, prior to the injecting step, the method comprises:

inserting the catheter into a compressed one of the blood vessels;

expanding expandable elements on the catheter to relieve the compression; and

inserting the needle through a vessel wall and into the intervascular space.

20. The method of item 19, wherein the injecting step comprises deploying the needle from a retracted position within the catheter, such as by using an actuator, to place a tip on the needle between the expandable elements.

As used herein, the following terms have the following meanings:

“A”, “an”, and “the” as used herein refers to both singular and plural referents unless the context clearly dictates otherwise. By way of example, “a compartment” refers to one or more than one compartment.

“About,” “substantially,” or “approximately,” as used herein referring to a measurable value, such as a parameter, an amount, a temporal duration, and the like, is meant to encompass variations of +/−20% or less, preferably +/−10% or less, more preferably +/−5% or less, even more preferably +/−1% or less, and still more preferably +1-0.1% or less of and from the specified value, in so far such variations are appropriate to perform in the disclosed invention. However, it is to be understood that the value to which the modifier “about” refers is itself also specifically disclosed.

“Comprise”, “comprising”, and “comprises” and “comprised of” as used herein are synonymous with “include”, “including”, “includes” or “contain”, “containing”, “contains” and are inclusive or open-ended terms that specifies the presence of what follows e.g. component and do not exclude or preclude the presence of additional, non-recited components, features, element, members, steps, known in the art or disclosed therein.

Although the invention has been described in conjunction with specific embodiments, many alternatives, modifications, and variations will be apparent to those skilled in the art. Accordingly, it embraces all such alternatives, modifications, and variations that fall within the appended claims' spirit and scope. All publications, patents and patent applications mentioned in this specification are herein incorporated in their entirety by reference into the specification, to the same extent as if each individual publication, patent or patent application was specifically and individually indicated to be incorporated herein by reference. In addition, or identification of any reference in this application shall not be construed as an admission that such reference is available as prior art to the present disclosure.

1. A catheter for relieving compression among blood vessels by introducing a filler into an intravascular space, comprising:
a shaft;
expandable elements supported by the shaft; and
a needle supported by the shaft, the needle, when deployed, including a tip located between the expandable elements for introducing the filler into the intravascular space.

2. The catheter of claim 1, wherein the shaft includes a lumen, and the needle is hollow and in communication with the lumen.

3. The catheter of claim 2, further including an actuator for deploying the needle from a retracted position within the lumen.

4. The catheter of claim 1, wherein the shaft includes an inflation lumen, and the expandable elements comprise inflatable balloons in fluid communication with the inflation lumen.

5. The catheter of claim 4, wherein the shaft comprises a guidewire lumen.

6. The catheter of claim 1, wherein a plurality of needles are provided, each having a tip located between the expandable elements.

7. The catheter of claim 1, wherein the needle is curved when deployed between the expandable elements.

8. The catheter of claim 1, wherein the needle comprises a shape memory material.

9. The catheter of claim 1, wherein the needle is adapted for delivering the filler from a remote source to a location in the intervascular space.

10. A system for relieving compression among blood vessels with an adjacent intervascular space, comprising:
a catheter including a needle adapted for extending into the intervascular space when deployed; and
a filler for delivery via the needle into the intravascular space for relieving compression among the blood vessels.

11. The system of claim 10, wherein the catheter includes a shaft having a distal end with spaced apart expandable elements, the needle when deployed including a tip located between the expandable elements.

12. The system of claim 11, wherein the expandable elements comprise inflatable balloons.

13. The system of claim 11, wherein the needle is curved when deployed.

14. The system of claim 11, wherein the needle comprises a shape memory material.

15. The system of claim 11, further including an actuator for moving the needle from a retracted position within the catheter to a deployed position for delivering the filler to the intervascular space.

16. The system of claim 11, wherein the catheter includes a plurality of needles.

17. A method of relieving compression among blood vessels, comprising:
injecting a filler into an intervascular space between the blood vessels.

18. The method of claim 17, wherein the injecting step comprises injecting the filler to the intervascular space using a needle connected to a catheter.

19. The method of claim 18, wherein, prior to the injecting step, the method comprises:
inserting the catheter into a compressed one of the blood vessels;
expanding expandable elements on the catheter to relieve the compression; and
inserting the needle through a vessel wall and into the intervascular space.

20. The method of claim 19, wherein the injecting step comprises deploying the needle from a retracted position within the catheter to a space between the expandable elements.
